(19) United States
(12) Patent Application Publication
(10) Pub. No.: US 2026/0020845 A1
(43) Pub. Date: Jan. 22, 2026

(54) Prediction and Tracking of Hormonal Changes Through Non-Invasive Physiological Measurements

(71) Applicant: Jano Life, Inc., Santa Clara, CA (US)

(72) Inventors: Anand Janefalkar, Milbrae, CA (US); Vasant Salgaonkar, Sunnyvale, CA (US); Tejas Mehta, Los Angeles, CA (US)

(21) Appl. No.: 19/244,871

(22) Filed: Jun. 20, 2025

Related U.S. Application Data
(63) Continuation-in-part of application No. 19/196,572, filed on May 1, 2025.
(60) Provisional application No. 63/672,676, filed on Jul. 17, 2024.
(60) Provisional application No. 63/703,006, filed on Oct. 3, 2024.

(51) Int. Cl. A61B 10/00; A61B 5/00; A61B 5/0245; A61B 5/0537; A61B 8/08

(52) U.S. Cl. CPC A61B 10/0012; A61B 5/0245; A61B 5/0537; A61B 5/4312; A61B 5/4875; A61B 5/7264; A61B 8/0825; A61B 2010/0016; A61B 2010/0019

(57) ABSTRACT

A series of electrodes and a machine learning system are configured to detect changes in biological tissue over time. Specifically, the system may include a wearable device configured to detect changes in tissue response to an electrical signal that may be indicative of cancerous tissue, e.g., breast cancer and/or other types of cancer. The system optionally combines measurements from bioimpedance sensors, miniaturized ultrasound arrays, temperature sensors, and/or printed microwave planar antenna to detect changes in breast tissue composition and vascularity. The system may also be used to detect other physiological states such as menstrual cycles and/or various conditions related to hydration or breathing patterns.

### CROSS-REFERENCE TO RELATED APPLICATIONS

This application is a Continuation-in Part of U.S. non-provisional patent application Ser. No. 19/196,572 filed May 1, 2025, which in turn claims benefit of and priority to U.S. provisional patent application Ser. No. 63/672,676 filed Jul. 17, 2024 and U.S. provisional patent application Ser. No. 63/703,006 filed Oct. 3, 2024, the disclosures of all of the above applications are hereby incorporated herein by reference.

### BACKGROUND

### Field of the Invention

The invention is in the field of physiological monitoring and in some embodiments the detection of hormonal changes using a wearable device.

### Related Art

The detection of breast cancer often depends on mammography, a technique subject to both false negative and false positive errors.

The detection of hormonal changes typically requires a blood or urine test. For example, common tests for pregnancy and fertility timing are blood or urine based.

### SUMMARY

A wearable electrode-based sensor system is configured for semi-continuous and long-term detection of conditions within a biological tissue. Specific applications are applied to the detection of cancer, such as breast cancer. The electrode-based sensor system is optionally combined with other types of sensors such as temperature, microwave imaging, and/or ultrasound sensors.

Other applications are applied to the monitoring of breast tissue as a way of detecting hormone cycles. For example, electrode-based sensors may be used to detect changes in breast composition and volume as a result in the menstrual cycle. These tissue changes occur because the volume of fibroglandular and ductal tissues increase during the luteal phase (post ovulation) of the menstrual cycle and decrease in the follicular phase leading to menses. Changes in the breast composition may be used in conjunction with other measurements, such as ultrasound, body temperature, heart rate (HR) and heart rate variability (HRV) to monitor hormones and related physiology.

Various embodiments of the invention include a cancer detection system comprising: an electrode array configured to be worn by a user in contact with the user's skin, the electrode array including a plurality of electrodes; a power source; a signal generator configured to apply electrical signals to one or more of the electrodes using the power source; a detector configured to detect electrical signals at one or more of the electrodes and to generate a digital signal output, the digital signal output being representative of a physiological state of a tissue of the user; control logic configured to activate the signal generator to generate a series of digital signal outputs over a period of time; memory configured to store the series of digital signal outputs; trained machine learning logic configured to detect a physiological state of the user that is indicative of breast cancer; and an I/O configured to communicate the digital signal output to the trained machine learning logic.

Various embodiments of the invention include a cancer detection system comprising: an electrode array configured to be worn by a user in contact with skin of the user, the electrode array including a plurality of electrodes; a power source (e.g., a battery); a signal generator configured to apply probe electrical signals to one or more of the plurality electrodes using the power source; a detector configured to detect response electrical signals at one or more of the plurality of electrodes and to generate digital signal outputs, the response electrical signals being responsive to the probe electrical signals and the digital signal outputs being representative of a physiological state of a tissue of the user; control logic configured to activate the signal generator to generate a series of the probe electrical signals over a period of time, each of the probe electrical signals resulting in at least one of the response electrical signals; memory configured to store the digital signal outputs; trained machine learning logic configured to detect a physiological state of the user based on the digital signal outputs, the physiological state being indicative of disease (e.g., cancer or breast cancer); and an I/O configured to communicate the digital signal output to the trained machine learning logic.

Various embodiments of the invention include a training system comprising: modeling logic configured to generate electrostatic models of breasts based on known tissue characteristics and breast structure data, wherein the tissue characteristics include characteristics of cancer tissue and a least two of: areola tissue, adipose tissue, epidermal tissue (skin), hypodermal fat, lactiferous ducts, and smooth muscle tissue; memory configured to store the electrostatic models; machine learning logic; and training logic configured to train the machine learning logic to detect the cancer tissue based on the electrostatic models and simulations of impedance measurements of one or two breasts as measured by a plurality of electrodes. The breast structure data optionally includes data representing at least 1000 breasts and optionally includes ultrasound data or microwave imaging data.

Various embodiments of the invention include a method of detecting cancer, the method comprising: optionally receiving a preliminary indication of cancer the preliminary indication being based on a mammogram or based on ultrasound data; providing a wearable sensor array including a plurality of electrodes, the plurality of electrodes being configured to provide electrical signals to a user's skin and to detect electrical signals on the user's skin; providing a series of electrical signals to members of the electrodes; detecting response signals at members of the electrodes; storing digital representations of the response signals in a memory; repeating the steps of providing a series of electrical signals, detecting response signals and storing digital representations of a period of time to create a series of digital signal outputs; providing the series of digital signal outputs to trained machine learning logic; and receiving an output of the machine learning logic, the output indicating that a change in the series of digital signal outputs is indicative of cancer.

Various embodiments of the invention include a method of training machine learning logic to detect cancer, the method comprising: receiving a model of a first human tissue, the model including electrical properties of the first human tissue; receiving a model of a second human tissue, the model including electrical properties of the second human tissue; generating a plurality of digital models of a human organ the plurality of models including at least the first and the second human tissue, wherein at least some of the digital models include a cancer; simulating responses of the digital models to electrical signals to generate a series of digital outputs; and training machine learning logic to detect the cancer using the series of digital outputs.

Various embodiments include a hormone tracking system comprising: an electrode array configured to be worn by a user in contact with skin of the user, the electrode array including a plurality of electrodes; a power source; a signal generator configured to apply probe electrical signals to one or more of the plurality electrodes using the power source; a detector configured to detect response electrical signals at one or more of the plurality of electrodes and to generate digital signal outputs, the response electrical signals being responsive to the probe electrical signals and the digital signal outputs being representative of a physiological state of a tissue of the user; control logic configured to activate the signal generator to generate a series of the probe electrical signals over a period of time, each of the probe electrical signals resulting in at least one of the response electrical signals; memory configured to store the digital signal outputs; trained machine learning logic configured to detect a physiological state of the user based on the digital signal outputs, the physiological state being indicative of water in a breast of the user; and an I/O configured to communicate the digital signal output to the trained machine learning logic.

Various embodiments include method of tracking a menstrual cycle of a user, the method comprising: optionally determining position of an array of electrodes on skin of the user, e.g., using a positioning structure and/or an electrical signal generated by the heart; sending probe electrical signals to skin of the user using an array of electrodes; detecting response electricals resulting from the probe electrical signals; processing the response electrical signals to detect hydration of tissue, e.g., breast tissue, of the user; repeating the steps of 1) sending probe electrical signals, 2) detecting response electrical signals, and 3) processing the response signals, determine timing of the menstrual cycle, e.g. a time of ovulation; and optionally detecting a variance in the time of the menstrual cycle to determine a physiologic state of the user, e.g. perimenopause or pregnancy. Optionally further using body temperature and/or heart rate is to determine the timing of the menstrual cycle in addition to the response signals. These methods are optionally adapted to tracking of hormone levels during pregnancy of the user. For example, non-invasive measurements of breast hydration using probe electrical signals may be used do deduce changes in hormone levels during pregnancy, these hormone levels may be indicative of a wide variety of medical conditions and may be indicative of a need for hormone augmentation.

### BRIEF DESCRIPTION OF THE DRAWINGS

FIG. 1 illustrates a cancer detection system, according to various embodiments of the invention.

FIGS. 2A-2C illustrates various sensor arrays, according to various embodiments of the invention.

FIG. 2D illustrates Impedance as a function of electrode position, according to various embodiments.

FIGS. 2E-2F illustrate transducer arrays, according to various embodiments of the invention.

FIG. 3A illustrates a model of a breast, according to various embodiments of the invention.

FIG. 3B illustrates a model of a breast in relation to a breast image, according to various embodiments of the invention.

FIG. 4 illustrates methods of detecting breast cancer using wearable sensors, according to various embodiments of the invention.

FIG. 5 illustrates a training system for training of a machine learning logic to detect cancer, according to various embodiments of an invention.

FIG. 6 illustrates methods of training a machine learning logic to detect breast cancer using electrode-based sensors, according to various embodiments of the invention.

FIG. 7 illustrates methods of tracking hormones and/or hydration using a wearable device, according to various embodiments of the invention.

FIG. 8 illustrates correlation between bioimpedance, temperature and menstrual cycle, according to various embodiments.

FIG. 9 illustrates changes in tissue impedance during a menstrual cycle, according to various embodiments.

### DETAILED DESCRIPTION

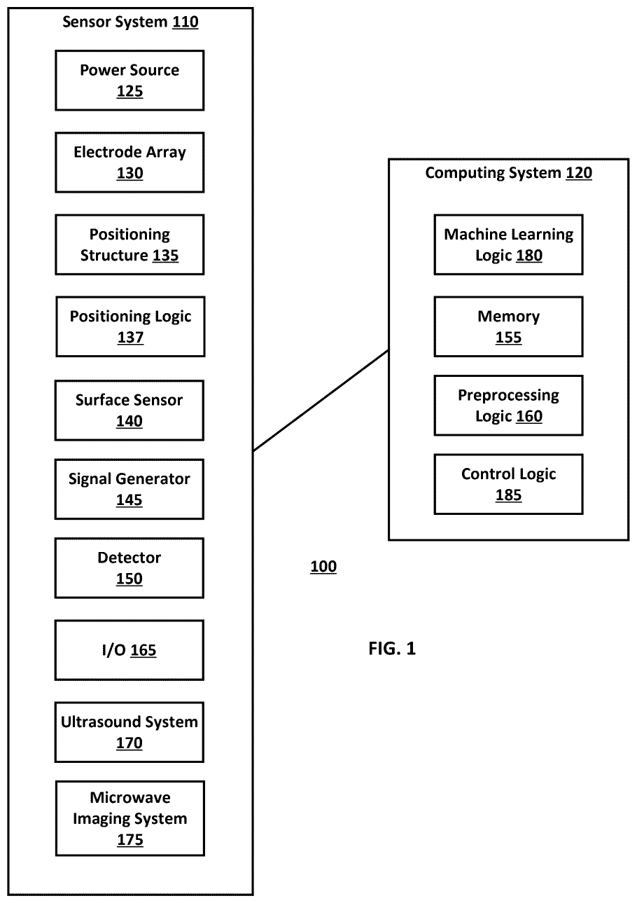

FIG. 1 illustrates a Detection System 100 comprising a Sensor System 110 and a Computing System 120. As discussed further elsewhere herein, Sensor System 110 includes a plurality of sensors configured to detect electrical characteristics of tissue, e.g., human breast tissue. Sensor System 110 is typically configured as a wearable device, e.g., a corset, a bra or bra insert. Computing System 120 can include one or more computing devices, optionally connected by a communication network (not shown). Specifically, Computing System 120 may include a mobile device such as a smartphone or tablet computer, and also a remote computing system including a cloud-based server. For example, Computing System 120 may include an application executing on a smartphone as well as a remote server. Sensor System 110 and Computing System 120 may be configured to communicate via any wired or wireless communication system. Any of the elements described herein as being included in Computing System 120 are alternatively or partially disposed within Sensor System 100. In some embodiments Computing System 120 includes a computing device configured to be implanted within a living organism. For example, under the skin of a person.

In various embodiments, Detection System 100 may be configured to detect, for example, Ductal Carcinoma In Situ (DCIS), Invasive Ductal Carcinoma (IDC), Invasive Lobular Carcinoma (ILC), Triple-Negative Breast Cancer, HER2-Positive Breast Cancer, and/or Inflammatory Breast Cancer (IBC). In alternative embodiments, Detection System 100 is configured to detect colorectal cancer, bladder cancer, pancreatic cancer, prostate cancer, lymphedema, lung cancer and/or the like.

Sensor System 110 is optionally disposed within a bra and/or bra insert. For example, Sensor System 110 may include a Power Source 125 and an Electrode Array 130 disposed in a bra insert and a Signal Generator 135 disposed in a bra. As used herein, a “bra insert” is meant to indicate a device configured to be inserted between an outer layer of a bra and the skin of a user, e.g., a person wearing the bra. A bra insert is typically removable from the bra. While a bra and bra insert and the detection of breast cancer are used as examples herein, the systems and methods discussed herein may be applicable to other articles of clothing and/or devices in contact with the skin of a user. For example, a post-mastectomy compression sleeve May include some or all of the electrodes of Electrode Array 130 configured to detect cancer in an auxiliary (arm pit) lymph node. The electrodes of Electrode Array 130 may be Ag, AgCl, -stainless steel, gold, Ni, and/or any other biocompatible metal or material.

Power Source 125 includes a source of electrical power configured to provide power to other elements of Sensor System 110. Power Source 125 can include an electrical power connector, a battery, a rechargeable battery, a capacitor, an electro-chemical cell, an inductive power coil, and/or the like. For example, in various embodiments, Power Source 125 includes a rechargeable battery and an inductive coil configured to wirelessly recharge the battery by receiving a radio frequency signal. Recharging may be accomplished by placing a breast insert including Power Source 125 in a wireless or wired charging device. Power Source 125 may include a USB (universal serial bus) or other standard type of electrical power connector.

In various embodiments, Power Source 125 is provided with a structural geometry specifically conducive to fit within a bra or bra insert. For example, Power Source 125 may include a battery having a thickness of less than 1, 2, 3 or 4 millimeters (or any range therebetween), and/or thickness to width ratio of at least 10, 20 or 30 to 1. Power Source 125 is optionally curved and/or flexible to conform to the curvature of a breast. Power Source 125 is optionally configured to fit within a bra strap and/or bra fastener. Power Source 125 is optionally at least 1 or 2 inches in length and configured to be disposed adjacent to a bra under-wire. For example, Power Source 125 may include a cylindrical battery at least 3 inches long and/or having a length to diameter ratio of at least 5, 10, 20 or 30 to 1. Power Source 125 is optionally further configured to function as a bra under-wire to support a breast.

Among other elements of Sensor System 110, Power Source 125 is configured to power Electrode Array 130 including a plurality of electrodes. Electrode Array 130 is configured to be worn by a user such that the electrode array is in electrical contact with the user's skin. For example, some embodiments include a single Power Source 125 configured to power electrodes in contact with both a right breast and also electrodes in contact with a left breast. Alternatively, some embodiments include a Power Source 125 having a first part configured to power electrodes in contact with a right breast and a second part configured to power electrodes in contact with a left breast. Electrode Array 130 may be worn by a user as a bra or as a bra insert place between a bra and the skin of the user, or with another item of clothing such as a jock strap, belt, sock, glasses, corset, pants, shirt, hat, shoe, bracelet, medical device, and/or the like. Optionally, Electrode Array 130 includes an adhesive or sticky surface to prevent movement of Electrode Array 130 on the user. “Contralateral” signals are signals that can compare the right and left breasts. For example, to compare changes that are the same in or different in each breast. Contralateral digital signals are digital signals that are derived from contralateral analog signals, e.g., response electrical signals or electro-cardio signals.

In some embodiments, Electrode Array 130 includes electrodes that are attached to the skin of a user, e.g., implanted or semi-permanently disposed in or on the skin. For example, Electrode Array 130 may include some electrodes included in bra and/or bra insert and some electrodes added to a user's skin as part of a conductive tattoo. In some embodiments, the electrodes include one or more body piercing. Electrodes attached to or within the skin, may be used as part of Positioning Structure 135. In such cases, positioning can be established by, for example, attachment to a piercing or making electrical contact between an electrode in a bra or bra insert and an electrode attached to the skin. Such electrodes may be used to make impedance measurements in areas of a user's body not normally covered by a bra or bra insert. For example, a conductive tattoo or other attached electrode may make electrical contact near axillary lymph nodes near the armpit or cervical lymph nodes near the neck of a user. Underwear, a jock strap or similar structure may be used to position electrodes near a user's prostate. A corset may be used to position electrodes near a user's pancreas. Contact between the skin of a user and an electrode may be direct or may be made via a conductor such as a body piercing or conductive adhesive. In one embodiment, contact is made via a nipple piercing. Electrodes may include copper, nickel, silver, steel, and/or any other suitable conductor.

In some embodiments positioning of Sensor System 110 includes detection of electrocardiogram (ECG) signal produced by the heart. While these signals are produced by the heart, they can be detected on the skin, the relative intensity and/or timing of the detected signals can depend on the position of the detecting electrodes within Electrode Array 130. Specifically, the relative intensities and/or timing of electrical signals from the heart can be used as a reference to deduce a position of Electrode Array 130 relative to a specific person's heart. Position relative to the heart is optionally determined using a computing logic, e.g., signal processing logic, statical analysis logic, and/or a trained machine learning logic disposed on Computing System 120. It is possible to map the structure of an ECG signal to specific positions and/or orientations of the electrodes on the chest. The output of this machine learning logic can, thus, include position, orientation and movement information regarding Electrode Array 130. In a simple example, an electro-cardio (e.g., ECG) signal detected using part of Electrode Array 130 may be used to distinguish which of two parts of Electrode Array 130 are placed on right and left breasts, respectively. An electro-cardio signal is a signal generated by, for example, electrical/nerve activity of the heart. The consistency of placement of Electrode Array 130 may also be detected by comparing detected ECG signals. ECG measurements may be used to measure heart rate (HR) and heart rate variability (HRV), e.g., through R-peak detection.

Further, changes in ECG signals as detected by Electrode Array 230, or other electrodes within Sensor System 110, can be used to detect relative motion between the heart and detecting sensors, which can be used to track and/or quantify movement. For example, breathing, walking, running, and/or other physical activities may be detected as relative movement between the heart and chest/breasts. Movement information may be combined with heart rate information to make further deductions regarding a person wearing Sensor System 110. In some embodiments ECG signals are used to determine electrode location relative to the heart.

In various embodiments, Electrode Array 130 includes at least 2, 3, 4, 8, 16 or 32 electrodes, or any range therebetween. These electrodes may alternatively be characterized as “sense,” “detection,” “probe,” “active,” “ground,” “signal,” “hot,” etc. depending on the role taken by a specific electrode in a particular measurement. For example, during a first measurement a first electrode may function as a ground or reference electrode, a second electrode may function as a probe or active electrode (configured to introduce an electrical signal into the skin), and a third electrode may function as a sense electrode configured to detect a result of the introduced electrical signal, each of the first second and third electrodes being in contact with a different location on a breast or other part of a user's body. In various embodiments, the electrodes of Electrode Array 130 may include a wide variety of sizes and shapes. For example, the electrodes may be round, oval, and/or rectangular. The electrodes May be characterized by a physical dimension of at least 2, 3, 4, 8, 16 or 32 millimeters (diameter), or any range therebetween. One embodiment includes electrodes of 6-7 mm in diameter. The electrodes may include dimples or micro needles configured to maximize skin contact. The electrodes may have thicknesses of less than 0.3, 0.5, 1, 2 or 3 millimeters (or any range therebetween). The electrodes may be curved or flexible to conform to the shape of a breast. In a specific example, Electrode Array 130 includes at least one ring electrode and/or at least one electrode at least 4 mm in diameter

Optionally, the roles of different electrodes are varied for different measurements. In various embodiments, Electrode Array 130 includes at least one ground electrode and one active electrode, at least one signal electrode and one sense electrode, etc. Electrode Array 130 may be configured such at least one pair of electrodes is disposed to detect an electrical signal (indicative of tissue impedance) between an areola and a location on a breast distal to the areola.

Electrode Array 130 may be disposed in two cups of a bra and/or (one or two) bra inserts, for the purpose of detecting cancer, e.g., breast cancer. For example, Electrode Array 130 may be distributed across two (right and left) bra inserts, each bra insert optionally including an electrode configured to be placed proximate to a nipple. Typically, data generated using such electrodes can be identified as being indicative of physiological conditions of the right and left breasts.

In some embodiments, at least one of the plurality of electrodes of Electrode Array 130 includes a ring electrode, e.g., an electrode having a ring shape. The ring electrode may be configured to be disposed over an areola and/or around a nipple. Such electrode may be part of a Positioning Structure 135, discussed further elsewhere herein.

The electrodes of Electrode Array 130 are configured to provide and/or detect electrical signals indicative of electrical impedance between electrodes through a user's tissue, e.g., breast tissue. Specifically, the electrodes may be configured to detect impedance through a user's nipple, areola, lactiferous duct, hypodermal fat, connective tissues, adipose tissues, smooth muscle, lymph nodes, sweat glands, and/or the like. The electrical impedance may vary as a function of signal frequency. As used herein the “frequency” of a signal is used to refer to a frequency of a waveform of the signal. For example, a signal may have a 100 Hz sign wave (a frequency of 100 Hz) or may include a periodic square wave, the square wave including a wide range of signal frequencies. (“Frequency,” is not meant to indicate how often a signal is applied, e.g., at a pulse rate of 10 Hz or once a day.) In some embodiments, the electrodes of Electrode Array 130 are configured to distinguish between surface impedance across a user's skin and bulk impedance through a user's tissues. Such distinction may be made based on signal propagation time, based on a dependance of impedance on signal frequency, and/or based on data generated by one or more Surface Sensor 140 (discussed further elsewhere herein.)

Sensor System 110 further includes a Signal Generator 145. Signal Generator 145 is configured to apply electrical signals to one or more of the electrodes of Electrode Array 130 using Power Source 125. These electrical signals may be referred to as probe electrical signals as they are intended to determine tissue characteristics, e.g., a physiological state of the tissue. The physiological state can include, for example, tissue type, hydration, salinity, fat percentage, presence of lipid bilayers, frequency dependent capacitance, frequency dependent inductance, three-dimensional structure, air content, blood, cell type, inflammation, and/or the like. Tissue types can include: cysts, calcifications, adenomas (not cancerous but abnormal) fatty tissue, membrane tissue, muscle tissue, cancerous tissue, non-cancerous tissue, abnormal tissue, epithelial tissue, nervous tissue, connective and/or any other type of biological tissue. The electrical signals may include a series of signals of varying signal frequency and/or varying intensity, e.g., voltage or current. For example, Signal Generator 145 may be configured to generate electrical signals of multiple frequencies, the multiple frequencies including a waveform including a first frequency and a signal at a second frequency, the second frequency being at least 2, 4, 8 or 10 times greater than the first frequency. The electrical signals can include frequencies of at least 10, 50, 100 or 200 kHz, or any range therebetween or determining extracellular impedance. The electrical signals can include frequencies of at least 0.5, 1, 2 or 3 MHz, or any range therebetween, for cell membrane impedance. Say 50 mA or less and less than 1 V. The waveforms can include complex waveforms, sinusoidal waveforms, sawtooth waveforms, square waves, multiple harmonics, symmetrical or asymmetrical waveforms, pulsed waveforms, various duty cycles, and/or the like.

Sensor System 110 further includes a Detector 150. Detector 150 is configured to detect electrical signals at one or more of the electrodes (of Electrode Array 130) and to generate a digital signal output responsive to the detected electrical signals. The detected (responsive) electrical signals are responsive to the probe electrical signals generated using Signal Generator 145 and applied to the tissue using Electrode Array 130. The digital signal output is representative of a physiological state of a tissue of the user, e.g., the presence of cancerous tissue, non-cancerous tissue, benign cysts, malignant tumors, fibroadenoma, tumor hypervascularity (blood flow), changes is breast density, and/or any of the other tissue types discussed herein.

In typical embodiments Detector 150 is configured to detect an impedance of the tissue between pairs of members of the plurality of electrodes. For example, Detector 150 may be configured to detect a voltage across the tissue and/or current through the tissue. Optionally, Detector 150 is configured to detect impedance as a function of electrical signal frequency. In such embodiments, Detector 150 may include an A/D (analog to digital) converter and memory configured to record and store a detected waveform including multiple frequencies.

In an illustrative example, Detector 150 is configured to detect voltage at a sense electrode relative to a voltage provided, using Signal Generator 145, at a probe electrode. The sense and probe electrodes being members of the plurality of electrodes. The rolls of these electrodes may change between measurements. Timing of the voltage detection may be based on a trigger signal received from Signal Generator 145. The detected voltage may be represented as a waveform over a period of time, e.g., over 0.1, 0.5, 1, 2, or 5 seconds, and may include multiple frequencies. In a specific example, if Signal Generator 145 is configured to provide a square wave signal to a probe electrode, Detector 150 may detect a waveform of voltage and/or current resulting from the square wave signal. In various embodiments, Detector 150 includes an A/D configured to generate 64, 128 or 256 bit data at frequencies of at least 100, 500, or 1000 Hz. For example, on one embodiment the AD allows for a 256-sample Depth (FIFO). As used herein “probe electrical signals” is used to refer to electrical signals applied to the skin (or other tissue) of a user in order to generate a detectable electrical response that can be used to deduce physiological information about the user. In contrast, “response electrical signals” and “sense electrical signals” are electrical signals detected at the skin of a user that are sensed and may be processed to generate physiological and/or other types of information. A specific electrode May both provide probe electrical signals and receive response electrical signals. Response electrical signals are a result of the probe electrical signals. More generally “sensed electrical signals” may include response electrical signals and signals not generated by probe electrical signals, e.g., electro-cardio signals or brainwave signals. For example, a response electrical signal May include a voltage difference between two electrodes of Electrode Array 130, the voltage at least in part being generated by (e.g., responsive to) a probe electrical signal applied using the same and/or different electrodes of Electrode Array 130.

Data generated by Detector 150 may be stored in Memory 155 and/or processed using Preprocessing Logic 160 as discussed further elsewhere herein. Elements of Memory 155 and Preprocessing Logic 160 may be included within Sensor System 110 and/or Computing System 120. The data generated by Detector 150 is typically communicated (preprocessed or not) from Sensor System 110 to Computing System 120 using an I/O (input/output) 165. I/O 165 and Memory 155 are discussed further elsewhere herein.

In some embodiments Signal Generator 145 and Detector 150 are configured to measure electrical signals between various members of Electrode Array 130. For example, Signal Generator 145 may be configured to provide an electrical signal at a ring electrode near a nipple and Detector 150 may be configured to alternatively detect signals at other members of Electrode Array 130, e.g., using a MUX to switch between sense electrodes. Each of the detected signals may be detected in different sampling events resulting from a separate probe signal.

Sensor System 110 may include any functional combination of the elements illustrated in FIG. 1. For example, a bra insert or bra may include Electrode Array 130, at least part of Power Source 125, Signal Generator 145, Detector 150, and/or I/O 165. These elements may be distributed between a bra and bra insert. For example, the Electrode Array 130 may be disposed in a surface of a bra while Power Source 125, Signal Generator 145, Detector 150 and I/O 165 are disposed within an insert configured to fit within a pocket of a bra, such that the surface of the bra is in contact with a wearer's breast when worn and the bra insert is removable from the pocket. Such embodiments allow the bra insert to be removed for washing of the bra. The wearer may be male or female.

In addition to electrostatic measurements made using Electrode Array 130, Sensor System 110 optionally includes other types of sensors. For example, Sensor System 110 may include an Ultrasound System 170 and/or a Microwave Imaging System 175. Ultrasound System 170 may include a plurality of ultrasound generation and detection elements disposed within the same bra or bra insert as Electrode Array 130 and configured to generate ultrasonic data. For example, Ultrasound System 170 may include an array of piezoelectric ultrasound generators and an array of ultrasonic microphones disposed to be in contact with a user's breast. Likewise, Microwave Imaging System 175 can include at least one microwave source and an array of microwave detectors configured to generate microwave data.

Optionally, and in contrast with imaging systems of the prior art, Ultrasound System 170 and Microwave Imaging System 175 need not be configured to generate sufficient data to generate a viewable image. Specifically, they may be configured to generate data that is indicative of changes in breast tissue, such as growth of cancer.

Ultrasound System 170 may operate via pulse-echo and/or through-transmission schemes. For example, an ultrasonic signal may be generated at one point on a user's breast and detected at another point on the user's breast, or an echo from a tissue boundary may be generated and detected at a same point on the breast. Ultrasonic signals are sensitive to both boundaries between structures and structure characteristics. For example, ultrasonic signals are sensitive to water content (hydration) within breast tissue. The high-fat regions have large acoustic attenuation and low speed of sound, while glandular volumes have lower acoustic attenuation but higher speed of sound. In some embodiments ultrasound frequencies between 0.5-3 MHz are utilized to detect tissue characteristics, e.g., hydration.

Ultrasound System 170 and Microwave Imaging System 175 are optionally configured to share Power Source 125 with any of the other elements of Sensor System 110. For example, Signal Generator 145 and Detector 150 may be configured to make daily measurements using a battery of Power Source 125, while Ultrasound System 170 and Microwave Imaging System 175 may be configured to make weekly measurements while an external power source is connected to Sensor System 110. Ultrasound System 170 may be configured to detect changes in blood flow using doppler ultrasound.

Computing System 120 includes trained Machine Learning Logic 180. Machine Learning Logic 180 is configured to detect a physiological state of the user that is indicative of disease, e.g., prostrate cancer or breast cancer. Machine Learning Logic 180 is configured to detect the physiological state based on data generated by Detector 150, Ultrasound System 170, and/or Microwave Imaging System 175, and optionally processed using Preprocessing Logic 160. Machine Learning Logic 180 is optionally trained using the systems and methods described elsewhere herein, e.g., in FIGS. 5 and 6.

Machine Learning Logic 180 optionally includes a plurality of neural networks and/or other AI, configured to perform different functions. For example, in some embodiments, part of Machine Learning Logic 180 is configured for detection of positions of Electrode Array 130 on the chest of a person, and/or of movement of Electrode Array 130 relative to a person's heart. In a specific example, Machine Learning Logic 180 may be used to give a person real-time feedback regarding positioning of Electrode Array 130 on the person's chest, and thus achieve a more optimum positioning. Machine Learning Logic 180 optionally includes a large language model (LLM), statistical logic, expert system, and/or the like.

Typically, Machine Learning Logic 180 is configured to detect the physiological state based on changes in a series of digital signal outputs received from Sensor System 110 over a period of time. Specifically, Machine Learning Logic 180 may be trained to receive data generated using Detector 150, Ultrasound System 170, and/or Microwave Imaging System 175 at different times over a 6 month or 1 year period, and based on changes in this data over time determine a physiological state of the user that is indicative of cancer. The time period over which data is collected may vary wildly in various embodiments. For example, data collected over 1, 3, 6, 12 and/or 24 months, or any range therebetween, may be used. Likewise, the data may be collected daily, weekly, and/or monthly.

In some embodiments, Machine Learning Logic 180 is configured to compare digital signal outputs received from Sensor System 110 that represents physiological states of right and left breasts. Such comparisons may detect changes that occur in one breast but not the other. Specifically, Machine Learning Logic 180 may be configured to detect cancer based on differences between data generated from members of the plurality of electrodes in contact with a right breast and digital signal outputs generated from members of the plurality of electrodes in contact with a left breast, and/or changes in these differences over time.

In a specific example, Computing System 120 may be configured to first receive a baseline of signals (baseline signals) generated using Sensor System 110 over an initial period of 1, 3 or 6 months. These baseline signals may identify normal time dependent variations in the physiological states of right and left breasts, e.g., variations resulting from a monthly hormone cycle. This baseline can then be used to normalize signals obtained at later times to compensate for natural time dependent variations. Machine Learning Logic 180 may then detect the physiological states using the normalized signals as input. Or, Machine Learning Logic 180 may be configured to compare the digital signal outputs with user specific baseline signals, wherein the user specific baseline signals are optionally time dependent, e.g., represent a one or more menstrual cycles. In some embodiments, Machine Learning Logic 180 is configured to compare physiological states by comparing data collected days or months apart. For example, growth of a suspected tumor may be monitored using measurements made on at least a daily, a weekly or a monthly basis.

The detection of physiological states using Machine Learning Logic 180 may be based on any combination of data generated using Detector 150, Ultrasound System 170 and Microwave Imaging System 175. Optionally, the trained machine learning system is configured to detect the changes indicative of in the physiological state by based on (optionally relative) signals from a first breast and a second breast. In some embodiments Machine Learning Logic 180 is further configured to detect the physiological state based on data generated using Detector 150, Ultrasound System 170 and/or Microwave Imaging System 175 over a period of time greater than 10 or 30 days and/or less than 30, 45, 90 or 180 days, and/or any range therebetween.

Computing System 120 further includes Control Logic 185. Control Logic 185 is configured to control various aspects of Sensor System 110 and Computing System 120. For example, Control Logic 185 may be configured to activate Signal Generator 145 to generate a series of digital signal outputs over a period of time. These digital signals may be coordinated with detection of electrical signals using Detector 150. Specifically, Control Logic 185 may be configured to use the signal generator to apply a series of probe electrical signals to Electrode Array 130, members of the series having different voltages and/or different frequencies. Each member of this series may be applied to different members of the electrodes in Electrode Array 130. Control Logic 185 is optionally configured to use Signal Generator 145 and Detector 150 to generate a first set of digital signal outputs from members of the plurality of electrodes in contact with a right breast and to generate a second set of digital signal outputs from members of the plurality of electrodes in contact with a left breast. Control Logic 185 is optionally further configured to control training of Machine Learning Logic 180, For example, using Training System 500 (FIG. 5) and/or the methods illustrated by FIG. 6. Control Logic 185 is optionally configured to communicate data processing results, collected data, and/or determined physiological states to a remote medical reporting or records system.

Control Logic 185 optionally includes an output configured to report a result from Machine Learning Logic 180 to a user or a medical professional. Control Logic 185 may include a user interface configured for a user to retrieve data or to enter information such as a user's age, the start of their menstrual cycle, and/or the like. Medical caregivers and patients (both examples of users) may have access to different user interfaces.

Control Logic 185 is typically configured to control timing of measurements made using Sensor System 110. For example, Control Logic 185 may be configured to make measurements using Electrode Array 130 on a daily basis and to make measurements using Ultrasound System 170 on a weekly basis. Control Logic 185 is optionally configured to control charging of Power Source 125. In some embodiments, Control Logic 185 is configured to increase the number of measurements made using Sensor System 110 based on initial digital data that might represent a tissue abnormality, e.g., cancer.

Memory 155 is configured to store data, a neural network of Machine Learning Logic 180, and/or computing instructions. For example, Memory 155 may be configured to store data generated by Detector 150, Ultrasound System 170 and/or Microwave Imaging System 175.

Memory 155 may further store processed instances of these data. For example, Memory 155 may be configured to store averaged waveforms recorded over several months and representing tissue impedance at different probe signal frequences as a function of a user's monthly hormone cycle. Memory 155 may be distributed amount a plurality of computing devices, and/or Sensor System 110. For example, an instance of Memory 155 disposed on Sensor System 110 may be configured to store raw digital data received from Detector 150 until this data can be transferred to Computing System 120 via I/O 165.

Sensor System 110 further includes I/O 165. I/O 165 is configured to communicate data and commends between Sensor System 110 and Computing System 120. For example, I/O 165 may be configured to communicate data generated using Detector 150, Ultrasound System 170 and Microwave Imaging System 175 to Computing System 120, wherein it may be used by Machine Learning Logic 180. I/O 165 may also be configured to receive instructions generated by Control Logic 185 for the control of Sensor System 110. For example, I/O 165 may be used to receive commands to activate Signal Generator 145 and Detector 150 to generate tissue impedance data; and/or to operate Ultrasound System 170 and/or Microwave Imaging System 175.

In various embodiments I/O 165 includes a radio frequency antenna, an inductive coil, an electro-optic, or an electrical connector. I/O 165 may be configured to communicate via Bluetooth, WiFi, NFC, Ethernet, USB, etc. I/O 165 is optionally further configured for charging Power Source 125. In a specific example, I/O 165 is configured to communicate with a portable computing device (such as a smartphone) or a remote computing device (such as a cloud-based server) via a communication network.

Sensor System 110 (or Computing System 120) optionally further includes Positioning Logic 137. Positioning Logic 137 is configured to determine positions of Electrode Array 130 based on signals received by electrodes therein. For example, in some embodiments, Positioning Logic 137 is configured to determine positions of the electrodes based on detection of electro-cardio signals (e.g., an ECG). The electro-cardio signals including signals produced by electrical activity of the heart (e.g., by nerves or muscles thereof). The electro-cardio signals may also include electrical signals generated by a pacemaker. Position may be determined based on the relative signals detected at different sense electrodes. In some embodiments, Positioning Logic 137 is configured to work in conjunction with Positioning Structure 135 to determine electrode positions. In some embodiments, Positioning Logic 137 is configured to determine which members (electrodes) of Electrode Array 130 are disposed on a right breast and which members of Electrode Array 130 are disposed on a left breast using electro-cardio signals by comparing the signals at each of these locations.

Sensor System 110 optionally further includes one or more Surface Sensor 140. Surface Sensor 140 is configured to detect temperature and/or humidity on a user's skin. For example, Surface Sensor 140 may include a plurality of thermocouples, resistance temperature detectors, blood oxygen sensor, blood pressure sensor (e.g., using pulse velocity), thermistors, semiconductor-based temperature sensors, infrared sensors (or other temperature detectors) distributed around a bra or bra insert. The temperature detectors may be configured to generate a skin temperature map of a breast. Such a skin temperature map may be indicative of blood flow within the breast. Surface Sensor 140 may include a capacitive humidity sensor, a resistive humidity sensor, a thermal conductivity humidity sensor or a surface acoustic wave humidity sensor. Surface Sensor 140 is optionally configured to couple with a body piercing, and thus sense conditions just below the skin.

Optionally, Machine Learning Logic 180 is further configured to detect physiological changes based on temperature and/or humidity data generated using the surface sensor. For example, Machine Learning Logic 180 may be configured to use a temperature map generated using Surface Sensor 140, and changes therein, to detect the physiological state of a breast, and thus the presence of cancer.

Computing System 120 optionally further includes Preprocessing Logic 160. Preprocessing logic 160 is configured to process the digital signal outputs received from Sensor System 110 prior to use by Machine Learning Logic 180. This processing of the digital signal outputs can include: applying a statistical function to the outputs (e.g., averaging), determining changes in the digital output signals over a time period, applying a Fourier or inverse Fourier or other transform to the data, discarding impedance data collected when the users skin was too moist as measured by Surface Sensor 140, classifying the digital signal outputs by electrode pairs, classifying the digital signal outputs by signal frequency, normalizing the digital signal outputs as a function of position of the electrode array, normalizing the digital signals using baseline data, normalizing the digital signal outputs as a function of signal amplitude, determining differences between digital output signals representative of right and left breasts, normalizing the digital output signals responsive to an output of the surface sensor, combining the digital output signals with ultrasound data and/or microwave imaging data, and/or any combination thereof. For example, Preprocessing Logic 160 may be configured to normalize data based on a baseline that varies over a user's monthly hormone cycle and then apply a Fourier transform to separate impedance measurements as a function of frequency. Parts of Preprocessing Logic 160 are optionally disposed in Sensor System 110. For example, averaging of data received from Detector 150 may occur in memory of Sensor System 110. The operation of Processing Logic 160 is typically controlled by Control Logic 185.

Sensor System 110 optionally further includes one or more Positioning Structure 135. Positioning Structure 135 is configured to position Electrode Array 130, Ultrasound System 170, and/or Microwave Imaging System 175 on a breast. Positioning Structure 135 is configured to position a bra or bra insert relative to one, two, three or more points on (each of) a user's breast. For example, Positioning Structure 135 can include an opening, indentation, and/or ring electrode configured to receive a nipple and, thus position Electrode Array 130 relative to an areola. Positioning Structure 135 may include attachment points to a bra underwire, bra cup, bra strap, bra clasp, and/or other points of a bra. These attachment points may include snaps, clips, magnets, and/or any other attachment device. In a specific example, Positioning Structure 135 includes an opening to accept a breast nipple and a clamp configured to attach a bra insert to a bra underwire or bra edge. Each bra cup or bra insert may have separate Positioning Structures 135.

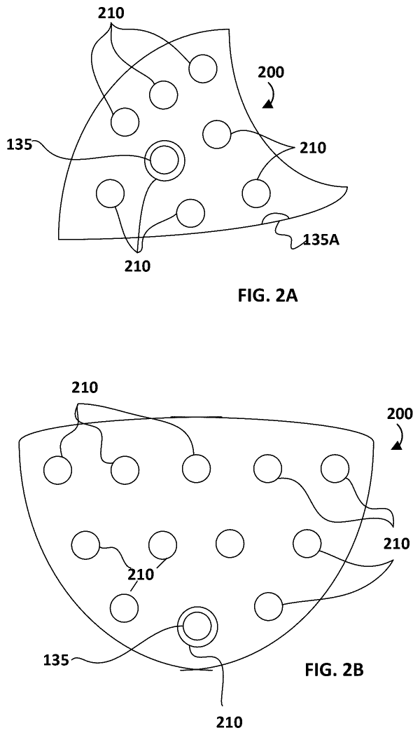

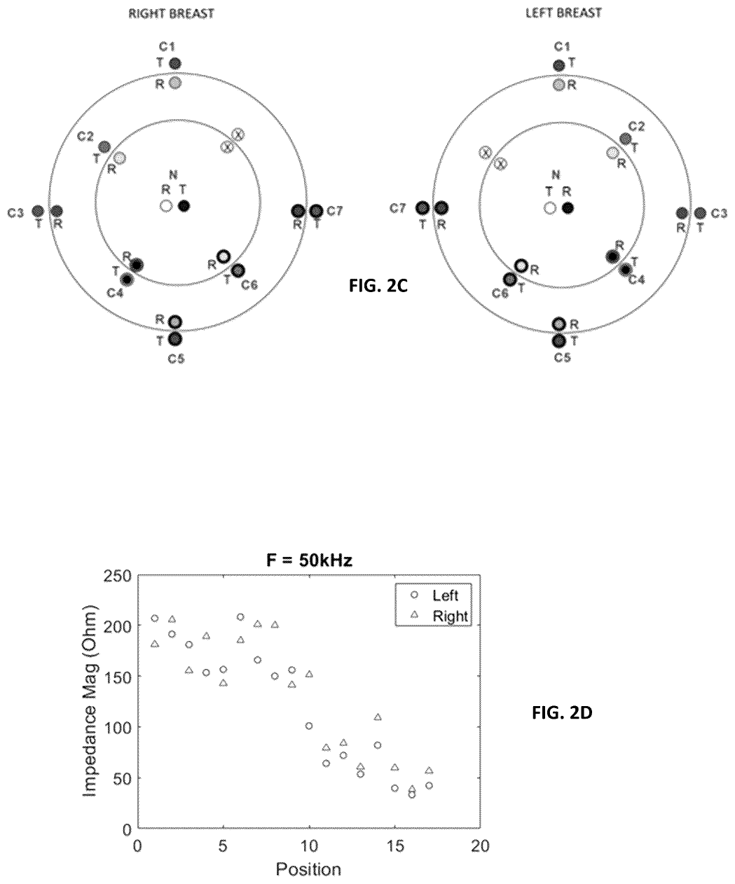

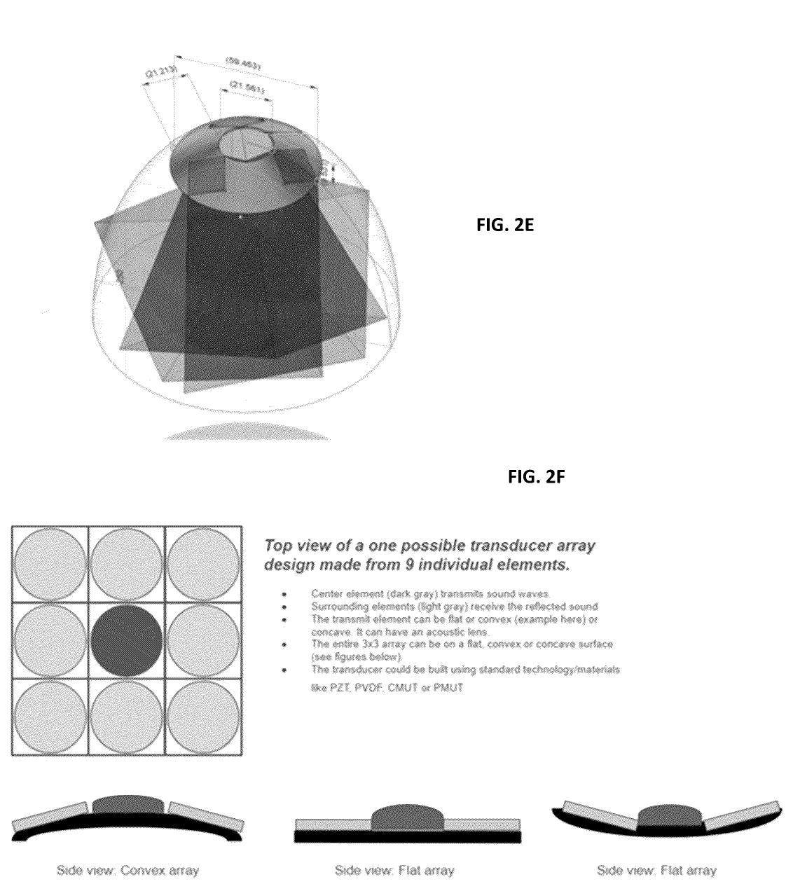

FIGS. 2A-2C illustrates various sensor arrays, according to various embodiments of the invention. FIG. 2A illustrates a Bra Insert 210 including Electrodes 220 and two Positioning Structures 135. Electrodes 220 are members of Electrode Array 130. One of the Positioning Structures 135 includes an indentation configured to accept a nipple and surrounded by a ring Electrode 220. The other Positioning Structure includes a Clip 135A configured to attached to a bra underwire. FIG. 2B illustrates a bra insert that may be used on either a right or left breast. FIG. 2C illustrates positions for transmit (T) and receive (R) electrodes on right and left breasts. Using the electrodes illustrated in FIG. 2C bioimpedance measurements are made using prototype bra inserts worn on the right and left breasts. Bioimpedance values are correlated with the position of the wearable sensors. High impedance is measured between wearable sensors C1-C5 that are far apart and near fatty regions of the breasts, (green circles). Low impedance is measured between wearable sensors C4-N that are close and near fibroglandular regions of the breasts, (black circles).

FIGS. 2E-2F illustrate transducer arrays, according to various embodiments of the invention. These transducers are optionally ultrasound or microwave transducers.

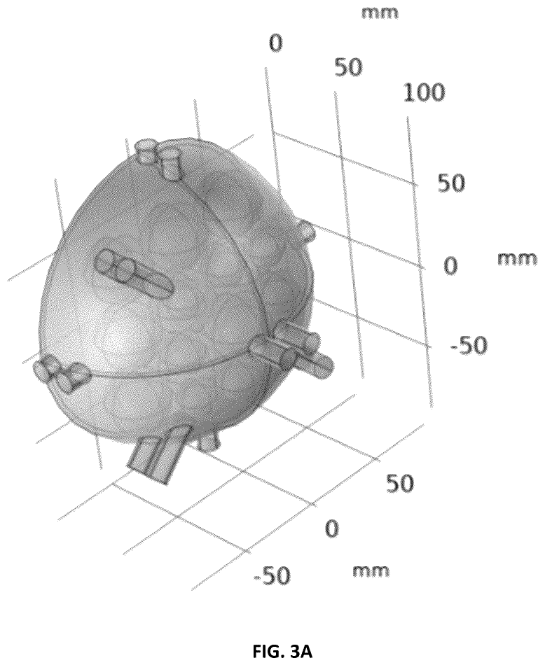

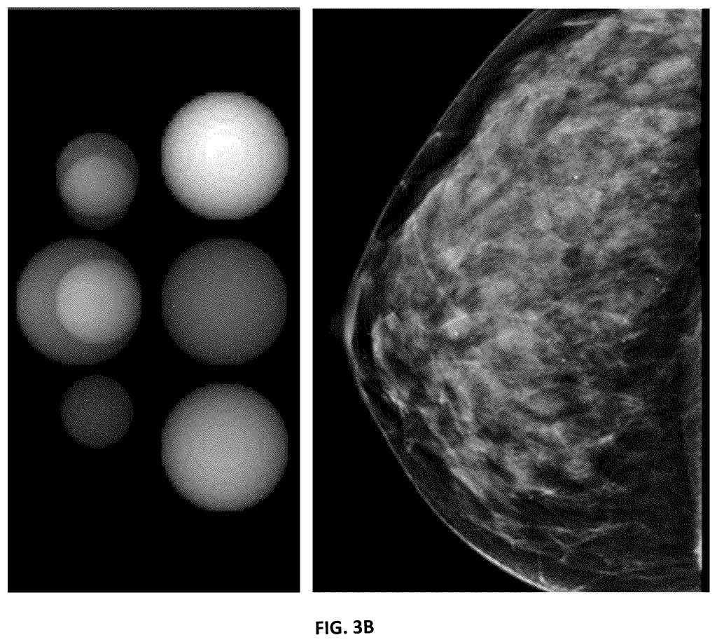

FIG. 3A illustrates a model of a breast, according to various embodiments of the invention. FIG. 3B illustrates a model of a breast in relation to a breast image, according to various embodiments of the invention.

In alternative embodiments, the Detection System 100 is adapted to operate as a non-invasive hormone tracking system and/or a system for monitoring tissue hydration, with or without also detecting cancer. Such hormone tracking is accomplished by tracking changes in a user's breasts, or other tissues, as a function of hormone levels. For example, changes in hormone levels may have a significant impact in tissue hydration and breast swelling. Different types of breast tissue hydrate differently resulting in further changes in breast tissue structure. In addition to breast tissue, adnominal tissue, facial tissue, ankle tissue, bones and joints, connective tissues, eyes, skeletal muscle, digestive tract, kidneys, etc. may change hydration in response to hormone cycles. In various embodiments, tissue hydration may be used as an indicator of hormone levels, optionally in conjunction with other data such as ultrasound data, body temperature and/or heart rate data.

System 100 can be used to detect changes in breast tissue composition (e.g., ratios between adipose/fat tissue, fibroglandular tissue and ductal tissue) based on values bioelectrical impedance or bioimpedance (BIA). BIA can be measured using electrical sensing electrodes (e.g., using Electrode Array 130 to make two point or four-point measurements). These impedance measurements are indicative of water content within the breast tissue (tissue hydration). Typically. high-fat tissues or regions have large impedance (low conductivity) and glandular volumes have lower impedance (high conductivity). Measurements can be made using micro-amp level electrical currents over a wide frequency range (for example low RF=5-250 kHz).

The tracking of hormones using System 100 can be used to monitor a woman's menstrual cycle, to monitor pregnancy, and/or to monitor post-partum hormone cycles. Such monitoring may have benefits such as detection of ovulation, detection of pregnancy, detection of perimenopause, and/or detection of abnormal hormone changes during or post pregnancy. Further, water detention (tissue hydration) may be a result of heart failure, kidney disease, liver disease, chronic lung diseases, thyroid disease, lupus, malnutrition, allergic reactions, hereditary angioedema, and certain medications. Any of these conditions may be tracked in embodiments in which System 100 is used to monitor tissue hydration. Further, tracking may be semi-continuous, with measurements optionally being made at least every 30, 60, 120 360, 720 or 1440 minutes, or any range therebetween.

In embodiments wherein System 100 is used to monitor hormones and/or tissue hydration, Machine Learning System 180 is adapted to generate an output representative of tissue hydration based on digital signal outputs received from Detector 150. Such digital signal outputs may be generated using Power Source 125, Electrode Array 130, Surface Sensor 140, Signal Generator 145, and/or Detector 150, as discussed elsewhere herein. Specifically, Machine Learning System 180 may be configured to detect a physiological state of the user based on the digital signal outputs, the physiological state being indicative of water in a breast (or other tissue) of the user or timing of the user's menstrual cycle. In various embodiments, Machine Learning System 180 is configured to output a value indicative of tissue hydration and/or a value indicative of an estimated hormone level. For such applications, Electrode Array 130 may be configured for attachment to a breast, wrist, head (e.g., as a pair of glasses), ankle, abdomen, finger (e.g., as a ring), neck, leg or arm, etc.

In various embodiments, the physiological state of the user is representative of ovulation of the user, a pregnancy (or lack thereof) of the user, a perimenopausal status of the user, and/or a menstrual cycle of the user. Specifically, the physiological state may be representative of a transition between a follicular phase and a luteal phase of a menstrual cycle of the user.

Optionally, Machine Learning System 180 is further configured to determine the physiological state based on output from other sensors, e.g., ultrasound sensors, temperature sensors, and/or a heart rate sensor included in System 100. A temperature sensor is optionally insulated from an ambient outside temperature by a bra or bra insert, thus being more representative of body/skin temperature than a non-insulated temperature sensor. A heart rates sensor may be used to measure heartrate (HR) and specific times and/or heart rate variability (HRV) of the user.

In some embodiments, Machine Learning System 180 is configured to detect a hydration state of the user, and is optionally able to distinguish between intercellular and extracellular hydration. Such distinction may be made through the use of different probe signal frequencies.

Depending on the physiological (e.g., hormonal) state determined, System 100 may further be configured to suggest hormonal supplements to be taken by the user in response to the response signals and output of Machine Learning System 180.

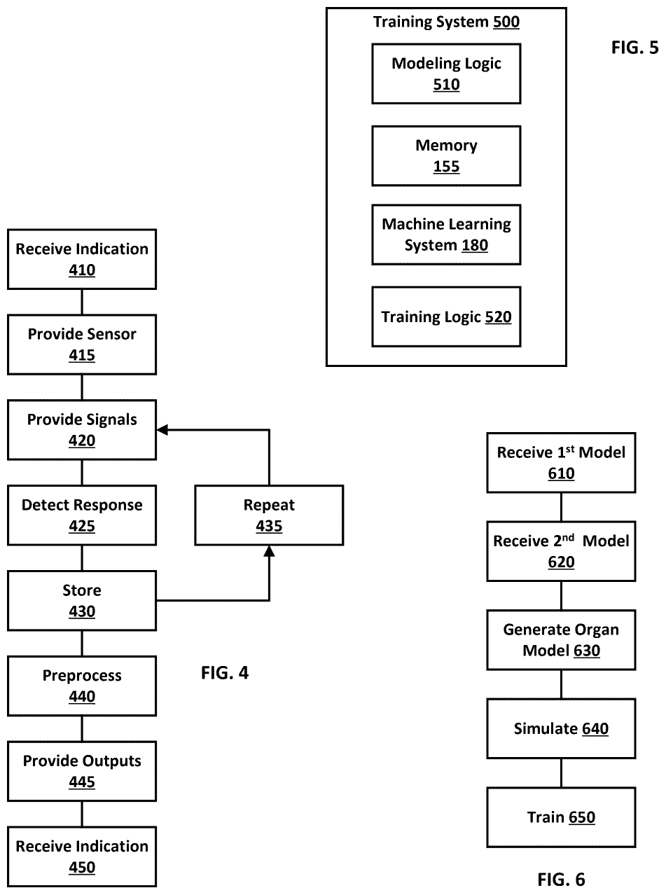

FIG. 4 illustrates methods of detecting breast cancer using wearable sensors, according to various embodiments of the invention. The method illustrated in FIG. 4 may be used as a screening tool for the detection of cancer, may be used as a monitoring tool for detection of a reemergence of cancer after treatment, and/or may be used as a follow-up after an initial test indicating possible of cancer or a preliminary indication of cancer. The wearable sensors can include electrode-based sensors, thermal sensors, ultrasound sensors, microwave sensors, and/or the like.

In an optional Received Indication Step 410, an indication of the presence of cancer is received. This indication can include for example, a DNA test, a biopsy, a mammogram, an ultrasound, or a microwave generated image. In a specific example, a mammogram may indicate a questionable feature in a breast that might be cancer. However, this diagnostic approach is well known to result in false positives. The methods of FIG. 4 may begin with such an initial indication followed by additional steps, as illustrated. The method illustrated in FIG. 4 is optionally performed using the systems of FIGS. 1, 2, and/or 3.

In a Provide Sensor Step 415 a wearable sensor system including a plurality of electrodes is provided to a user. For example, Electrode Array 130 may be provided as part of a Bra, Bra insert, and/or other wearable. The plurality of electrodes being configured to provide probe signals to a user's skin and to detect resulting electrical signals on the user's skin.

In a Provide Signals Step 420, a series of electrical signals are provided to members of the plurality of electrodes. These signals are optionally generated using Signal Generator 145 under control of Control Logic 185. As described elsewhere herein, the provided waveforms can include a wide variety of waveforms. Optionally, the waveforms are altered to generate specific information in response to data previously obtained. For example, a first output of the Machine Learning Logic 180 be used to Control Logic 185 to modify the probe signal to obtain further information.

In a Detect Response Step 425 signals response to the electrical signals is detected at members of the plurality of electrodes. The response signals may be indicative of tissue impedance of breast tissue, optionally as a function of frequency. The detected signals may be detected using Detector 150 and include signals from a variety of combinations of the plurality of electrodes. Detect Response Step 425 may include sampling at different pairs of electrodes and converting a voltage or current detected between each pair to a digital representation using an A/D converter.

In a Store Step 430 digital representations of the response signals are stored in a memory, e.g., Memory 155. The representations may be stored in Sensor System 110 and/or Computing System 120. Store Step 430 optionally includes communication of the response signals or the digital representations from Sensor System 110 to Computing System 120 via I/O 165.

In an optional Repeat Step 435 the steps of providing a series of electrical signals (420), detecting response signals (425) and storing digital representations (430) are repeated. These steps may be repeated over a period of time to create a series of digital signal outputs. As described elsewhere herein, multiple series of measurements may be made over periods of weeks or months, each series including measurements between multiple pairs of electrodes.

In an optional Preprocess Step 440 the digital signal outputs are preprocessed, e.g., using Preprocessing Logic 160. For example, the stored data may be averaged and/or data collected when the user's skin is too moist may be disregarded. Or, the digital signal outputs may be normalized for variations in the locations of electrodes on a user's breasts. Any of the preprocessing discussed elsewhere herein may be included in Preprocess Step 440.

In a Provide Outputs Step 445 the stored digital representations are provided to a machine learning system, e.g., to Machine Learning Logic 180. Typically, the machine learning system is trained to detect indications of cancer in the digital representations. For example, as discussed elsewhere herein, the machine learning system may be configured to detect breast cancer based on changes in the response signals over weeks or months, and/or based on differences in changes between right and left breasts.

In an optional Receive Indication Step 450 an output of Machine Learning Logic 180 is received. The output of Machine Learning Logic 180 may include an indication of the presence of cancer in the user, e.g., patient. This indication may be provided to the user or a caregiver thereof via a user interface on a computing device. Specifically, the output may indicate that a change in the series of digital signal outputs is indicative of cancer. This indication is optionally assigned a quantitative score. For example, the output may include a probability of 95%, 90%, 80%, 75%, 50% (or any range therebetween) that cancer is present. The output may also include a suggestion of a location within a specific breast.

This output may be based on the data provided in Provide Outputs Step 445, and/or any of the other data discussed herein. For example, ultrasound or microwave imaging data, and/or data generated by Surface Sensor 140. In some embodiments, the output of Machine Learning Logic 180 is further based on clinical data regarding the user. For example, a history of the user having a specific type of cancer, mammogram results, a family history of cancer, obesity, DNA profile, and/or the like may be included as an input to Machine Learning Logic 180. The output may be representative of a user's response to therapy, such as radiation, viral or chemotherapy.

FIG. 5 illustrates a training system for training of a machine learning system, e.g., Machine Learning Logic 180, to detect cancer, according to various embodiments of an invention. Training System 500 is configured to train a machine learning system, e.g., Machine Learning Logic 180, to detect cancer. Training System 500 may use data generated using an electrode array worn by a person, e.g., as part of a bra or bra insert, and/or may use synthetic data based on a physiological model. In some embodiments, Training System 500 is configured to first train a machine learning system using synthetic data generated using the physiological model and then to further train the machine learning system based on baseline data representative of a specific user. In these embodiments, the machine learning system is optionally multi-layered. For example, a base layer of Machine Learning Logic 180 may be trained using synthetic data and a secondary layer may be trained using data generated using Sensor System 110. The secondary layer may then be used as an input and/or output layer to the base layer.

In a specific example, a base layer of Machine Learning Logic 180 may be trained using synthetic data that is not specific to a particular user. Sensor System 110 may then be used to gather baseline data specific to an individual. This baseline data may then be used to train the secondary layer specific to the individual. Once the machine learning system is trained for cancer detection, digital output generated using Signal Generator 145 and Detector 150 may be provided to inputs of the base layer, the output of the base layer is provided to inputs of the secondary layer and an output of secondary layer may be used as an indication of cancer in the individual. Alternatively, the secondary layer may receive the digital output and the output of the secondary layer is provided to the base layer. The machine learning system may include more than two layers, e.g., input and output layers trained to a specific individual and a generic base layer.

The machine learning system, or a base layer thereof, may be trained using synthetic data specific to a particular set of user characteristics. For example, the synthetic data may be generated to represent a person having two breasts or only one breast, for breasts within a specific size range, for a person having breast implants, for a person having DNA markers for an increased risk of breast cancer, for a person having undergone cancer treatment, and/or for monitoring of lymph nodes. Specifically, an instance of the machine learning system may be trained to detect recurrence of cancer and/or cancer in lymph nodes

As illustrated in FIG. 5, Training System 500 includes Modeling Logic 510. Modeling Logic 510 is configured to generate electrostatic models of breasts based on known tissue characteristics and breast structure data. The tissue characteristics can include, for example, characteristics of cancer tissue and a least two of: areola tissue, adipose tissue, cysts, adenomas, calcifications, hypodermal fat, lactiferous ducts, and smooth muscle tissue. Breast structure data is commonly available from clinical data, so models of breasts found in the human population can readily be generated.

Training Logic 520 is configured to generate synthetic data based on the models of breasts by simulating electrical signals within the models. The simulated electrical signals are dependent on the tissue characteristics found in a particular model, including characteristics of cancer tissue. Typically, the synthetic data is generated using a wide variety of breast models representative of actual breasts. To generate a representative sample of synthetic data, at least 1000, 100,000, 1 million, 10 million or 50 million breast models may be considered. The synthetic data may further include ultrasound data or microwave imaging data. In such embodiments, Modeling Logic 510 is further configured to generate the models of breasts based on known ultrasonic and/or microwave characteristics of the tissues, and the training logic is further configured to train the machine learning system to detect the cancer tissue based on ultrasound data and/or microwave imaging data. In various embodiments, the synthetic data may represent (electrostatic) models of at least 1, 2, 4 or 8 types of cancer tissue.

Training logic 520 is further configured to train the machine learning system to detect cancerous tissue based on the electrostatic models and simulations of impedance measurements of one or two breasts as measured by a plurality of electrodes, e.g., Electrode Array 130. For example, all or part of Machine Learning Logic 180 may be trained using the synthetic data generated using Training Logic 520, the synthetic data being based on the tissue characteristics and breast structure data.

An instance of Memory 155 is optionally included in Training System 500. This instance of Memory 155 may be configured to store the synthetic data, Machine Learning Logic 180, Modeling Logic 510, and/or Training Logic 520.

FIG. 6 illustrates methods of training a machine learning system, e.g., Machine Leaning Logic 180, to detect breast cancer using electrode-based sensors, according to various embodiments of the invention. These methods are optionally performed using the Training System 500 illustrated in FIG. 5. While breast cancer is presented as an illustrative example, the methods of FIG. 6 may be used to train machine learning systems to detect other types of cancers or abnormal tissue in other organs such as the prostate, lungs, liver, nervous system, cervix, colon, and bladder.

The training methods illustrated in FIG. 6 include a Receive 1^{st} Model Step 610 in which a first model of a first tissue is received and a Receive 2^{nd} Model Step 620 in which a second model of a second tissue is received. The first and/or second tissues optionally include any of the types of cancerous or non-cancerous tissues discussed herein. They also include breast implants, such as silicone breast implants. The first and second models typically include electrical properties of the respective tissues. These properties can include impedance at various signal frequencies.

In some embodiments, Steps 610 and 620 further include receipt of tissue characteristics related to ultrasound transmission and/or microwave imaging.

In a Generate Organ Model Step 630 a model of a human organ is generated. The generated organ model is based on at least the first and second tissue models. Typically, a breast model may include more than two types of tissue models in order to realistically represent a human breast. For example, a model may include various types of cancer tissue and/or various types of non-cancerous tissue, including any of the tissue types discussed herein. The organ model typically represents approximations of actual organs as determined from clinical data. Specifically, the organ model may include the presence of specify tissues in specific places as would be expected from studies of actual breasts and/or other organs.

In a Simulate Step 640 responses of the organ model to electrical signals are simulated to generate a series of digital outputs, e.g., to generate synthetic training data. Steps through 640 are typically repeated a large number of times with different organ models to generate a set of synthetic training data the is representative of the various organs, e.g., breasts, expected to be found in a population. At least some of the organ models include cancer tissue such that the resulting data can be used to train a machine learning system to identify the presence of cancerous tissue in a breast.

In some embodiments, Simulate Step 650 further includes simulation of the organ models to generate synthetic ultrasound and/or microwave imaging data.

In a Train Step 650 the synthetic data generated in Step 610 through 640 is used to train a machine learning system, such as Machine Learning Logic 180. The trained machine learning model can then be used to detect cancer as described elsewhere herein. Following training using the synthetic data, Train Step 650 optionally includes further training of all or part of the machine learning system using baseline data obtained from a specific user.

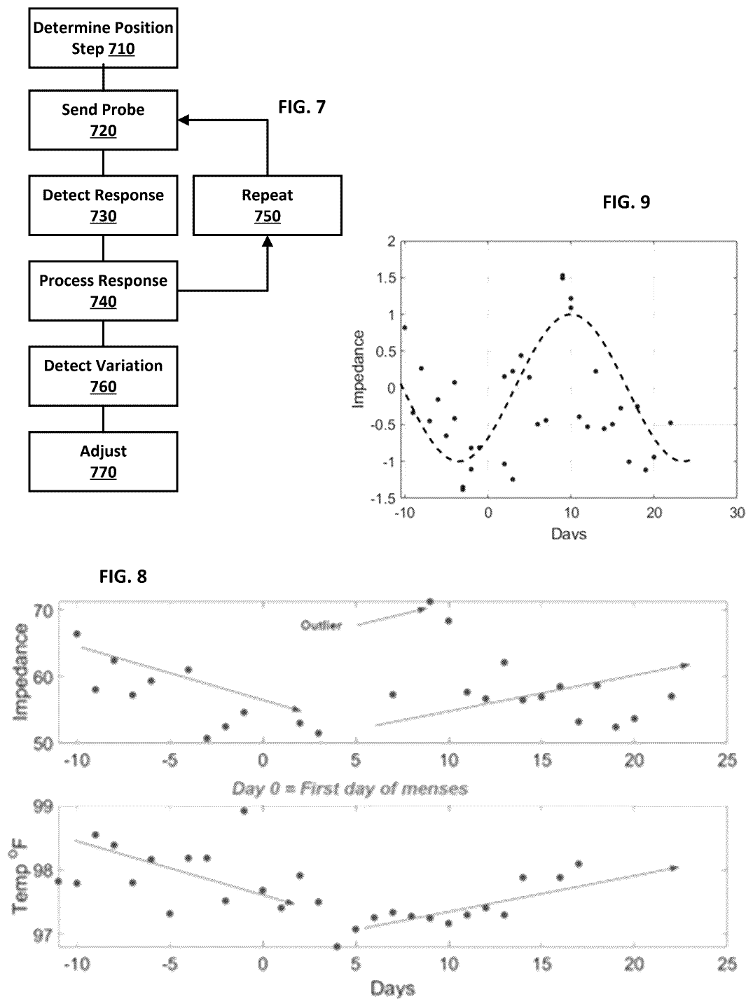

FIG. 7 illustrates methods of tracking hormones and/or hydration using a wearable device, according to various embodiments of the invention. These methods are optionally performed using System 100 as illustrated in FIG. 1. Tracked hormones may be used to infer any of the medical conditions and/or physiological states discussed herein. The methods illustrated by FIG. 7 are optionally performed in conjunction with other methods described herein.

In an optional Determine Position Step 710, positions of electrodes (e.g., Electrode Array 130) on a user are determined. Such determination is optionally performed using one or more Positioning Structure 135. Alternatively, the positions of electrodes may be determined by sensing an electrical signal generated by the user's heart, e.g., an electrocardiogram. The electrical signals may be used to determine electrode positions relative to the heart.

In a Send Probe Step 720 probe electrical signals are applied to the skin of the user using the electrodes, e.g. using Electrode Array 130. The skin may include skin of the breast or any of the other body parts discussed herein. For example, the electrical signals may be applied bilaterally to both breasts of the user. As described elsewhere herein, the applied electrical signals can be of various frequencies, voltages and may be applied in various pulse patterns. Different signal frequencies may be used to measure different impedances.

In a Detect Response Step 730 response signals resulting from the probe electrical signals are detected. The response signals are optionally detected using the same electrodes within Electrode Array 130 as were used to apply the probe electrical signals. Detect Response Step 730 may be performed using Detector 150 and result in a digital signal output. Optionally, Detect Response Step 730 is accompanied by additional steps (not shown) in which ultrasonic, heart rate, breathing, blood O₂, and/or temperature data are collected.

In a Process Response Step 740 the digital signals generated by Detector 150 are processed, for example using Machine Learning Logic 180. The result of this processing can include, for example, an estimated hydration level, an estimated hormone level, and/or the like. Machine Learning Logic 180 may be configured to estimate hormone level, either a number or a categorical range such as low, normal or high. Machine Learning Logic 180 may also be configured to return information on the phase of the hormonal cycle such as follicular or luteal or high-fertility window or onset of menses on a monthly basis. Over a longer time periods, the Machine Learning Logic 180 may also be configured to make predictions about deviations from regular hormonal cycles due to pregnancy, perimenopause, hormone therapy, medication, conditions such as endometriosis, etc.

The digital signals may be pre-processed using several techniques that increase the robustness and accuracy of Machine Learning Logic 180 predictions. These can include techniques such as temporal and spatial averaging, mathematical methods such as first or second order derivatives, or complex feature extraction techniques such as principal component analysis (PCA), singular value decomposition (SVD), t-distributed Stochastic Neighbor Embedding (t-SNE), etc.

Optionally Process Response Step 740 is preceded by a pre-processing step (e.g., Pre-Process Step 440) as discussed elsewhere herein.

In a Repeat Step 750 steps 720, 730 and 740 are repeated over time. For example, they may be repeated every 30 minutes or hourly over a period of weeks or months. In some cases, these steps are repeated for at least 3 menstrual cycles as desired. The first few menstrual cycles may then be used to establish a baseline to which later measurements can be compared.

In an optional Detect Variation Step 760 the user's hormone and/or hydration data may be fitted to an expected pattern, e.g., an expected menstrual cycle. Such fitting may be, for example, to a sign or cosign function. Once a fitting is made, it can be used to represent an expected time variance of the user's physiological state. This model may be compared with future measurements made using the methods of FIG. 7 to identify variations from the user's expected pattern. Such variation may indicate a need for medical intervention (e.g., a blood test or change in treatment), or a changed physiological state (e.g., pregnancy, kidney failure, or perimenopause, etc.).

In an optional Adjust Step 770, hormone treatments and/or other medications given to the user are adjusted based on the results of Process Response Step 740 and/or Detect Variation Step 760. For example, if the user is found to be accumulating water in the breasts or ankles, ademia medication may be adjusted by qualified medical person. In another example, if the user's menstrual cycle does not transition from the luteal phase to follicular phase when expected, the user may be given a pregnancy test.

FIG. 8 illustrates correlation between bioimpedance, temperature and menstrual cycle, according to various embodiments. As illustrated, both body temperature and electrical impedance (as measured at a user's breasts) vary over their menstrual cycle. The fit to the illustrated lines can be improved by making more frequent measurements, e.g., more frequent than would be practical via blood tests. Impedance is displayed in relative/normalized units. The illustrated tissue changes occur because the volume of fibroglandular and ductal tissues increase during the luteal phase (post ovulation) and decrease in the follicular phase leading to menses.

FIG. 9 illustrates changes in tissue impedance during a menstrual cycle, according to various embodiments. The values illustrated were obtained using the system illustrated in FIG. 1 and have been fit, for the purpose of example, to a sinusoidal curve. The approach can be modified to use parametric functions (hyperbola, parabola, rectified sine waves), polynomial functions (X-square x-cube, etc.), and regression approaches (multi-variate, logistic, splines, etc.). The input to these models can be other measured physiological parameters such as basal temperature, heart rate, heart rate variability, breathing patterns, blood oxygen, other symptoms and metabolic indicators (self-reported or measured using other platforms).

Several embodiments are specifically illustrated and/or described herein. However, it will be appreciated that modifications and variations are covered by the above teachings and within the scope of the appended claims without departing from the spirit and intended scope thereof. For example, while distinguishing between right and left breasts is provided herein as an example, the systems and methods discussed herein are also applicable to a person having only one breast, e.g., a woman have had a partial mastectomy. The systems and methods discussed herein may be applied to the detection of other cancers or other medical conditions. Further, while the detection of cancer is used herein as an example, the systems and methods describe are alternatively used to detect other medical conditions, such as fatty liver, kidney store, bladder stones, respiratory or cardiac function. Thus, the use of Sensor system 110 is not limited to the breasts or chest, but may be used elsewhere on the body and/or inside the body, e.g., as a stent, catheter or endoluminal device.

In some embodiments, Sensor System 110 and Computing System 120 are configured to detect breathing patterns. For example, Machine Learning Logic 180 may be configured to detect and process breathing patterns based on digital electrical signals received from Detector 150, e.g., based on response signals that vary with expansion and contraction of the chest. These embodiments may be used to monitor conditions in which breathing patterns are indicative of a physiological state, e.g., sleep apnea, sleep cycles, asthma, labored breathing, hyperventilation, hypoventilation, anxiety or COPD. Any of the embodiments discussed herein may be adapted to include breathing analysis. In the detection of breathing patterns, tissue measurements are optionally made at least 3, 5, 10 or 20 times in a normal breathing cycle of about 6 seconds. In some embodiments ECG and breathing data are generated contemporaneously. For example, holding one's breath can impact the ECG data. Determining the breathing cycle by detecting chest motion (and changes in impedance thereby) can therefore allow for improved processing and understanding of ECG data. Alternatively, ECG data may be used to confirm breathing data. Breathing patterns are optionally compared to the output of a blood oxygen sensor, e.g., pulse oximeter, included in Sensor System 110, e.g., as part of a bra or bra insert.

The embodiments discussed herein are illustrative of the present invention. As these embodiments of the present invention are described with reference to illustrations, various modifications or adaptations of the methods and or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

Computing systems and/or logic referred to herein can comprise an integrated circuit, a microprocessor, a personal computer, a server, a distributed computing system, a communication device, a network device, or the like, and various combinations of the same. A computing system or logic may also comprise volatile and/or non-volatile memory such as random access memory (RAM), dynamic random access memory (DRAM), static random access memory (SRAM), magnetic media, optical media, nano-media, a hard drive, a compact disk, a digital versatile disc (DVD), optical circuits, and/or other devices configured for storing analog or digital information, such as in a database. A computer-readable medium, as used herein, expressly excludes paper. Computer-implemented steps of the methods noted herein can comprise a set of instructions stored on a computer-readable medium that when executed cause the computing system to perform the steps. A computing system programmed to perform particular functions pursuant to instructions from program software is a special purpose computing system for performing those particular functions. Data that is manipulated by a special purpose computing system while performing those particular functions is at least electronically saved in buffers of the computing system, physically changing the special purpose computing system from one state to the next with each change to the stored data. The terms “machine learning system” and “machine learning logic” used herein are meant to include artificial intelligence systems, neural networks, a generative pretrained transformer, and/or the like.

The “logic” discussed herein is explicitly defined to include hardware, firmware or software stored on a non-transient computer readable medium, or any combinations thereof. This logic may be implemented in a quantum, electronic and/or digital device (e.g., a circuit) to produce a special purpose computing system. Any of the systems discussed herein optionally include a microprocessor, including quantum, electronic and/or optical circuits, configured to execute any combination of the logic discussed herein. The methods discussed herein optionally include execution of the logic by said microprocessor.

What is claimed is:

1. A hormone tracking system comprising:
an electrode array configured to be worn by a user in contact with skin of the user, the electrode array including a plurality of electrodes;
a power source;
a signal generator configured to apply probe electrical signals to one or more of the plurality electrodes using the power source;
a detector configured to detect response electrical signals at one or more of the plurality of electrodes and to generate digital signal outputs, the response electrical signals being responsive to the probe electrical signals and the digital signal outputs being representative of a physiological state of a tissue of the user;
control logic configured to activate the signal generator to generate a series of the probe electrical signals over a period of time, each of the probe electrical signals resulting in at least one of the response electrical signals;
memory configured to store the digital signal outputs;
trained machine learning logic configured to detect a physiological state of the user based on the digital signal outputs, the physiological state being indicative of hydration a breast of the user; and
an I/O configured to communicate the digital signal output to the trained machine learning logic.

2. The system of claim 1, wherein the physiological state is representative of ovulation of the user.

3. The system of claim 1, wherein the physiological state includes a pregnancy status of the user.

4. The system of claim 1, wherein the physiological state includes a perimenopausal status of the user.

5. The system of claim 1, wherein the physiological state is representative of a menstrual cycle of the user.

6. The system of claim 5, wherein the physiological state is representative of a transition between a follicular phase and a luteal phase of the menstrual cycle of the user.

7. The system of claim 5, further comprising a temperature sensor, wherein the trained machine learning logic is further configured to detect the physiological state using a temperature of the user measured using the temperature sensor.

8. The system of claim 5, wherein the trained machine learning logic is further configured to detect the physiological state using a heart rate or heart rate variability of the user measured using the electrode array.

9. The system of claim 1, further comprising a ultrasound system, wherein the trained machine learning logic is further configured to detect the physiological state using ultrasound data generated using the ultrasound system.

10. The system of claim 1, wherein the trained machine learning logic is configured to detect the physiological state during pregnancy or lactation of the user.

11. The system of claim 1, further including at least one positioning structure configured to position the electrode array on a breast or further including positioning logic configured to detect a position of the electrode array based on detection of electro-cardio signals.

12. The system of claim 11, wherein the positioning structure is configured to position the electrode relative to an areola.

13. The system of claim 11, wherein the positioning structure includes a connection to a bra.

14. The system of claim 1, wherein at least one electrode of the electrode array is a ring electrode disposed around a positioning structure.

15. The system of claim 1, wherein at least one electrode of the electrode array is configured to detect response electrical signals indicative of impedance through a nipple, areola or lactiferous duct.

16. The system of claim 1, wherein the electrode array is configured to be distributed in two cups of a bra or two bra inserts, and the detector is further configured to generate digital signal outputs that distinguish between response electrical signals generated from first and second breasts.

17. The system of claim 16, wherein the bra or the bra inserts include the electrode array, at least part of the power source, at least part of the signal generator and at least part of the detector.

18. The system of claim 1, wherein the trained machine learning logic is configured to detect changes in the series of digital signal outputs over the period of time, wherein the changes are indicative of a change in the physiological state of the user that is indicative of a menstrual cycle, perimenopause, menopause, endometriosis, fibroids or a pregnancy of the user.

19. The system of claim 1, wherein the machine learning logic is configured to compare digital signal outputs generated from members of the plurality of electrodes in contact with a right breast to digital signal outputs generated from members of the plurality of electrodes in contact with a left breast.

20. The system of claim 1, further comprising preprocessing logic configured to process the digital signal outputs, the processing of the digital signal outputs including: classifying the digital signal outputs by electrode pairs, classifying the digital signal outputs by signal frequency, normalizing the digital signal outputs as a function of position of the electrode array, or determining changes in the digital output signals over a time period.
